(19) United States
(12) Patent Application Publication
(10) Pub. No.: US 2005/0272098 A1
(43) Pub. Date: Dec. 8, 2005

(54) Quantitation of endothelial microparticles

(76) Inventor: Anthony F. Tramontano, Franklin Square, NY (US)

Correspondence Address: DILWORTH & BARRESE, LLP, 333 EARLE OVINGTON BLVD., UNIONDALE, NY, 11553, US

(21) Appl. No.: 11/113,786

(22) Filed: Apr. 25, 2005

Related U.S. Application Data
(60) Provisional application No. 60/564,856, filed on Apr. 23, 2004.

(51) Int. Cl. G01N 33/53; G01N 33/567

(52) U.S. Cl. 435/7.2

(57) ABSTRACT

The present invention provides a method for determining the presence of atherosclerosis in a patient and a method for measuring atherosclerotic burden in a patient diagnosed with atherosclerosis. The methods use a two-color flow cytometric technique to quantitate the absolute number of endothelial microparticles (EMPs) in vitro using a known quantity of labeled beads such as TruCount™ beads, as an internal standard. Also provided are kits useful for practicing the methods of the present invention.

### CROSS REFERENCE TO RELATED APPLICATIONS

This application claims the benefit of U.S. Provisional Application Ser. No. 60/564,856, filed Apr. 23, 2004, which application is incorporated by reference herein.

### BACKGROUND OF THE INVENTION

Atherosclerosis is a condition characterized by elevated inflammatory cytokines and endothelial cell (EC) dysfunction.[1] Recently, it has been shown that ECs release endothelial microparticles (EMPs) on activation and that elevated serum levels of EMPs are demonstrable in patients with thrombotic disorders including coronary artery disease (CAD).[2] In addition to indicating EC injury, EMPs likely play a direct role in thrombogenesis by virtue of tissue factor, platelet factor-3 activity and a distinctive antigenic profile of cellular adhesion molecules (CAM) that may preferentially bind to and activate leukocytes.[3] In vitro, ECs release EMPs when activated by inflammatory cytokines, such as tumor necrosis factor-α (TNF-α).[4] Thus, EMP release reflects EC activation, and analysis of EMPs can serve as a marker of EC activation.

HMG-CoA reductase inhibitor (statin) therapy targeted at lowering of atherogenic low-density lipoprotein can significantly reduce cardiovascular morbidity and mortality.[5] Furthermore, studies with statins have demonstrated that this survival benefit precedes any significant reduction in serum cholesterol levels suggesting an additional effect on the ECs, independent of cholesterol reduction. Among the non-lipid lowering actions of statins are plaque stabilization, reduced inflammation, reversal of endothelial dysfunction, and decreased thrombogenicity.[6]

In ECs, the Rho family of small GTPases are key regulators of both cell adhesion and the cytoskeleton.[7] Interestingly, the anti-inflammatory properties of statins may be mediated by inhibition of Rho protein isoprenylation, thus preventing membrane attachment of Rho proteins and the subsequent activation of downstream effectors such as Rho-kinase (ROK).[8]

Presently, cardiovascular risk assessment is based on established risk factors including gender, age, smoking, diabetes, and serum concentration of cholesterol. There are only a few markers presently available for the detection of endothelial activation ex vivo. The present invention provides methods for determining the presence of clinical atherosclerosis in a patient and also for measuring atherosclerotic burden in patients diagnosed with atherosclerosis. The methods are reproducible and require only a small amount of sample, thus lending themselves to the testing of clinical specimens.

### SUMMARY OF THE INVENTION

Elevated plasma levels of endothelial microparticles (EMPs) are associated with the presence of clinical atherosclerosis. In accordance with the present invention, the absolute number of EMPs may be enumerated using a novel two-color flow cytometric immunostaining technique employing containers such as tubes, having a known number of solid surfaces, such as beads, which are labeled with a fluorescent dye.

The present invention therefore provides a method for determining the presence of clinical atherosclerosis in a patient. The method comprises the steps of obtaining a plasma sample from a patient; centrifuging the plasma sample in order to collect a pellet of cellular debris containing endothelial microparticles (EMPs); resuspending the pelleted debris with an appropriate buffer; adding both labeled antibodies against cellular adhesion molecules (CAMs) that are specific to EMPs and the resuspended pellet to a container having a known number of solid surfaces wherein the solid surfaces are labeled with a fluorescent dye; performing FACScan flow cytometry on the prepared sample in order to calculate the absolute number of EMPs therein; and correlating an increased level of EMPs in the sample derived from the patient (compared to a corresponding control sample), with the presence of clinical atherosclerosis. Preferably, the control sample is age matched, and derived from a healthy individual.

In another aspect of the invention, there is provided a method for measuring atherosclerotic burden in patients diagnosed with atherosclerosis. The method comprises the steps of obtaining a plasma sample from a patient diagnosed with atherosclerosis; centrifuging the plasma sample in order to collect a pellet of cellular debris containing endothelial microparticles (EMPs); resuspending the pelleted debris with an appropriate buffer; adding both labeled antibodies against cellular adhesion molecules (CAMs) that are specific to EMPs and the resuspended pellet to a container such as a tube, having a known number of solid surfaces, such as beads, which are labeled with a fluorescent dye; and performing FACScan™ flow cytometry on the prepared sample in order to calculate the absolute number of EMPs (atherosclerotic burden) therein.

The present invention further provides kits for determining the presence of atherosclerosis in a patient or for measuring atherosclerotic burden in a patient. A kit comprises a labeled antibody against cellular adhesion molecules (CAMs) specific to EMPs and a container having a known number of solid surfaces wherein the solid surfaces are labeled with a fluorescent dye. A kit may further comprise a container for collecting blood from a patient and/or preparing a plasma sample. Instructions for its use may also be included in the kit.

### BRIEF DESCRIPTIONS OF THE DRAWINGS

FIG. 1 is a representative CELLQuest™ document showing dot plots and position of regions used to identify EMP and TruCount™ bead populations. Labels indicate the gate used to display the data. TruCount™ Tube Beads are in Black. 1 and 2 μm calibration beads are in blue. EMP are in red. A and B: TruCount™ beads=R1, 1 and 2 μm beads=R2. C: The number of EMP were enumerated by using known density TruCount™ beads. D: PE-conjugated anti-CD31.

FIG. 2A is a representative western blot showing the effect of fluvastatin on RhoA activation.

FIG. 2B graphically depicts quantitative analysis with densitometry. Similar results were obtained in three independent experiments.

### DETAILED DESCRIPTION OF THE INVENTION

Currently, cardiovascular risk assessment is based on established risk factors, including gender, age, smoking, diabetes, and serum concentrations of cholesterol. At present, there are only a few markers for the detection of endothelial activation ex vivo. The methods of the present invention for quantitation of EMP levels thus serve as an early diagnostic screening tool for CAD in addition to measuring atherosclerotic burden in the management of patients with known CAD.

A difficult problem in EMP studies has been comparing results between laboratories. Since EMPs exist as heterogeneous species in proportions varying with the nature of the EC injury, it is unlikely that any single marker will efficiently label total EMPs. Thus the need for precise and reproducible quantification is necessary if EMPs are to be a reliable marker for atherosclerosis.

The present invention provides a new, two-color flow cytometric technique to quantitate the absolute number of EMP in vitro with labeled beads such as TruCount™ beads, as an internal standard. This technique is reproducible and requires a small amount of sample, thus lending itself to the testing of clinical specimens. The methods employ plasma samples, free of red blood cells. Since there are no red blood cells in the plasma samples, a lysing buffer is not needed.

The present invention therefore provides a method for determining the presence of clinical atherosclerosis in a patient. The method comprises the steps of obtaining a plasma sample from a patient; centrifuging the plasma sample in order to collect a pellet of cellular debris containing endothelial microparticles (EMPs); resuspending the pelleted debris with an appropriate buffer; adding both labeled antibodies against cellular adhesion molecules (CAMs) that are specific to EMPs and the resuspended pellet to a container having a known number of solid surfaces wherein the solid surfaces are labeled with a fluorescent dye; performing FACScan flow cytometry on the prepared sample in order to calculate the absolute number of EMPs therein; and correlating an increased level of EMPs in the sample derived from the patient (compared to a corresponding control sample), with the presence of clinical atherosclerosis. Preferably, the control sample is age matched, and derived from a healthy individual.

In another aspect of the invention, there is provided a method for measuring atherosclerotic burden in patients diagnosed with atherosclerosis. The method comprises the steps of obtaining a plasma sample from a patient diagnosed with atherosclerosis; centrifuging the plasma sample in order to collect a pellet of cellular debris containing endothelial microparticles (EMPs); resuspending the pelleted debris with an appropriate buffer; adding both labeled antibodies against cellular adhesion molecules (CAMs) that are specific to EMPs and the resuspended pellet to a container such as a tube, having a known number of solid surfaces, such as beads, which are labeled with a fluorescent dye; and performing FACScan™ flow cytometry on the prepared sample in order to calculate the absolute number of EMPs (atherosclerotic burden) therein.

The methods of the present invention may be used in assessing any type of clinical atherosclerosis including but not limited to: diabetes mellitus, coronary artery disease (CAD), and acute coronary syndrome.

A plasma sample may be obtained using methods well known in the art. For example, blood may be drawn from a patient following standard venipuncture procedure. Plasma may then be obtained from the blood sample following standard procedures including but not limited to, centrifuging the blood sample at about 1,500×g for about 15-20 minutes, followed by decanting or pipeting of the plasma layer.

In order to collect the EMPs, a plasma sample may be centrifuged in a range of from about 15,000 to about 20,000×g. Preferably, the plasma sample is ultra centrifuged at around 17,570×g at a temperature of about 4° C.

Different buffers may be considered appropriate for resuspending the pelleted cellular debris which contains the EMPs. Such buffers include reagent grade (distilled or deionized) water and phosphate buffered saline (PBS) pH 7.4. Preferably, PBS buffer (Sheath fluid) is used.

EMPs contain the antigens CD62E, CD105, CD106, CD51, CD31, and CD54 but do not have the antigen CD42A or CD42b. Methods of labeling antibodies are well known in the art. Preferably, the antibodies against CAMs are FITC-conjugated and/or PE-conjugated. Examples include monoclonal anti-human CD62E-FITC, CDC105-FITC, CD51-FITC, CD106-PE, CD31-PE, and CD54-PE, available through Ancell Co. (Bayport, Minn.).

The solid surfaces are preferably beads. Since EMPs have a diameter of roughly 1-2 μM, the beads for use in the present invention should have a diameter larger than 2 μM. Preferably, the beads are between 3 and 5 μM in diameter. More preferably, the beads are about 4 μM in diameter. Beads may be made of different materials, including but not limited to glass, plastic, polystyrene, and acrylic.

The total number of beads per container or tube should be known ahead of time. In addition, the beads are preferably fluorescently labeled. In a preferred embodiment, fluorescent beads are those contained in TruCount™ tubes, available from Becton Dickinson Biosciences, (San Jose, Calif.).

Methods of flow cytometry are well known in the art. See e.g., (1976) Herzenber et al. (1976) Sci. Amer., 234:108. Becton Dickinson Biosciences (San Jose, Calif.) makes several flow cytometers and associated software and hardware, including FACScan™ brand flow cytometer, and CELLQuest™ software, useful in practicing the methods of the present invention. EMPs may be excited with 488 nm light and logarithmic green and red fluorescences of FITC and PE may be measured through 530/30 nm and 585/42 nm bandpass filters, respectively. In accordance with the present invention, the absolute number of EMPs (atherosclerotic burden) may be calculated from the appropriate dot plot values entered into the following formula:

$$Absolute number of EMP \times {10}^{6} / mL = \frac{\# of events in EMP region ( R2 )}{\# of beads collected ( R1 )} \times \frac{total \# beads per tube}{test volume ( 50 μl )}$$

The present invention further provides kits for determining the presence of atherosclerosis in a patient or for measuring atherosclerotic burden in a patient. A kit comprises a labeled antibody against cellular adhesion molecules (CAMs) specific to EMPs and a container having a known number of solid surfaces wherein the solid surfaces are labeled with a fluorescent dye. A kit may further comprise a container for collecting blood from a patient and/or preparing a plasma sample and/or an appropriate buffer for resuspending debris pelleted from the plasma sample . Instructions for its use may also be included in the kit. Preferably, the labeled antibody contained in the kit is at least one of monoclonal anti-human CD62E-FITC, CDC105-FITC, CD51-FITC, CD106-PE, CD31-PE, or CD54-PE.

The following examples further illustrate the present invention and are not meant in any way to limit the scope thereof.

### EXAMPLE 1

### Materials and Methods

Culture products were from Cambrex Bio Science (Walkersville, Md.). TNF-α was purchased from Sigma (St. Louis, Mo.). Rho-kinase inhibitor (Y-27632) and Fluvastatin were purchased from CalBiochem. Fluorescent TruCount™ bead lyophilized pellets were from Becton Dickinson Biosciences (San Jose, Calif.). Calibration beads were obtained from Molecular Probes (Eugene Oreg.). Monoclonal anti-human CD62E-FITC, CD105-FITC, CD51-FITC, CD106-PE, CD31-PE, CD54-PE antibodies were obtained from Ancell Co. (Bayport, Minn.). Rhotekin Rho Binding Domain agarose, GTPγS and GDP were obtained from Upstate Biotechnology (Lake Placid, N.Y.). RhoA antibody was purchased from Santa Cruz Biotechnology (Santa Cruz, Calif.). 8-16% SDS-PAGE gel was obtained from (Cambrex, Rockland, Me.). HRP-conjugated Goat anti-rabbit IgG was obtained from Cell Signaling (Beverly, Mass.).

Cell Culture and EMP Preparation

HCAECs were purchased from Cambrex Bio Science (Walkersville, Md.) and cultured according to the supplier's specifications. All experiments were performed on confluent cells. EC were treated with 10 ng/mL TNF-α alone or with the addition of fluvatstain (0.1 μmol/mL) or ROK inhibitor, Y-27632 (10 μmol M/L). Controls were un-treated. After 24 hours culture supernatants from flasks containing HCAEC were collected and centrifuged for 30 min at 17,570×g, (4° C.). Pellets were re-suspended in 1 mL of PBS and were analyzed on the same day.

Flow Cytometric Analysis

For single platform method, 3 μL each of FITC-conjugated and PE-conjugated monoclonal antibodies against the above mentioned CAMs plus 50 μL of re-suspended pellet were added to tubes (two antibodies/tube) preloaded with fluorescent TruCount™ bead lyophilized pellets (Becton Dickinson Biosciences, San Jose, Calif.). Tubes were incubated for 20 min at room temperature. Analysis of EMPs was performed using a FACScan Flow Cytometer (Becton Dickinson Biosciences, San Jose, Calif.). The EMPs were excited with 488 nm light from a 15 mW argon laser. Logarithmic green and red fluorescences of FITC and PE were measured through 530/30 nm and 585/42 nm bandpass filters, respectively. Data from 2,000 events were acquired and analyzed with the use of CELLQuest™ software (version 3.3, Becton Dickinson). The absolute number of EMPs was calculated from the appropriate dot plot values entered into the following formula:

$$Absolute number of EMP \times {10}^{6} / mL = \frac{\# of events in EMP region ( R2 )}{\# of beads collected ( R1 )} \times \frac{total \# beads per tube}{test volume ( 50 μL )}$$

The total number of beads per tube is supplied by the manufacturer and varies among lot numbers.
RhoA Activity

RhoA activity was determined by a pull-down assay. The cell lysates were incubated with Rhotekin Rho Binding Domain agarose for 45 minutes. The agarose beads were collected and electrophoresed in 8-16% SDS-PAGE gel (Cambrex, Rockland, Me.). GDP and GTP-labeling of cell lysates served as negative and positive controls, respectively. Western blotting was performed with RhoA antibody at a dilution of 1:200. The blot densities were analyzed with Mocha Image Analysis (version 1.2.10).

Statistical Analysis

Statistical analysis was performed using SPSS 10.0.7 statistical software (SPSS Inc., Chicago, Ill.). Values are stated as mean±SEM. One-way ANOVA was used to compare means from three or more groups. In cases where the data were not normally distributed, the Mann-Whitney rank sum test was used. A value of p<0.05 was considered as significant for all tests.

### EXAMPLE 2

### Results

Quantitation of EMP

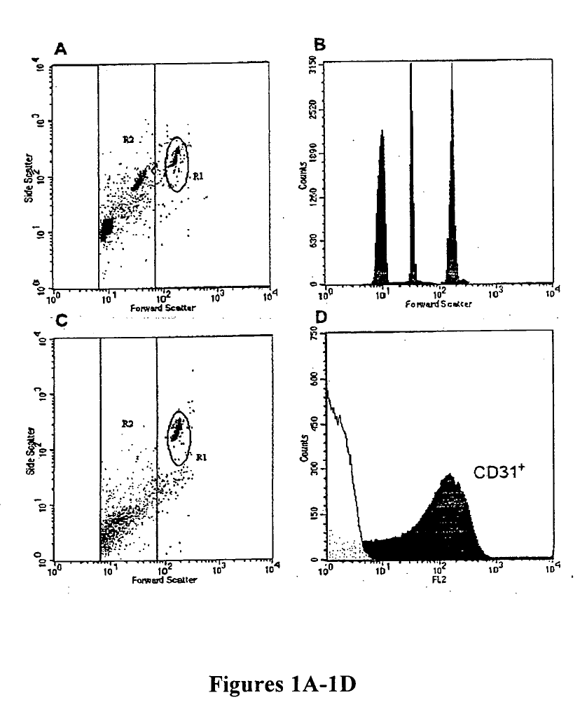

EMP are sub-microscopic membranous particles (size range about 1.5 μm), which are shed from TNF-α-activated ECs.[10] We defined EMP as elements ranging in size between 1 and 2 μm as assessed by their logarithmic amplification of the FSC and SCC signals (FIG. 1). To evaluate the number of EMP released, two-color flow cytometric immunostaining of prepared samples from HCAEC culture was performed. Bivariant FSC/SCC efficiently distinguished calibration beads of 1 and 2 μm in R2 from TruCountTM beads that were scattered in R1. (FIGS. 1A and B). The number of TruCountTM beads in addition to EMP count was enumerated from the R1 and R2 regions respectively (FIG. 1C). The purity as well as the proportion of EMP in this gate was identified further using FITC/PE fluorochrome-conjugated monoclonal antibodies to the above mentioned CAMs. A representative histogram is shown for CD31⁺ EMPs (FIG. 1D).

Release of CAM-Specific EMP in TNF-α-Activated HCAEC

TNF-α is a pivotal inflammatory cytokine in patients with hypercholesterolemia. [11] In addition, TNF-α generates the release of EMPs from EC in vitro.[4] Data presented in Table1show that EC release species of EMP exhibiting qualitative and quantitative differences in antigenic phenotypes in response to activation.

As assessed by flow cytometry, EMP specific for CD31, CD105, CD51, CD54, CD62E, CD106 were detectable from cultured HCAEC. Treatment of HCAEC with TNF-α (10 ng/mL) for 24 hours significantly increased the total number (EMP×10⁶/mL) of CD105, CD31 and CD51 specific EMP (CD105: 43±4.7 to 124±34, CD31: 145±36 to 233±77, CD51: 29.8 to 76.4±46, p<0.05). CD62 and CD54 positive EMP increased by 15% and 57% respectively, however, this was not significant. Levels of CD106 specific EMPs remained unchanged after treatment with TNF-α.

Statin and ROK Inhibitor Suppresses TNF-α-Induced EMP Release

Fluvastatin has been shown to have anti-thrombotic properties.[12] Table 1 shows that fluvastatin (0.1 μL) significantly suppresses the release of CD105⁺ and CD51⁺ EMP (124±34 to 45±7.6 and 76.4±46 to 43 ±7.3, p<0.05). Treatment with Y-27632 (10 μL) reproduced the effect of fluvastatin demonstrated by a significant decrease in CD105⁺ and CD31⁺ EMP (124±32 to 56±13, and 76±26 to 44±10, p<0.05). CD31³¹ and CD54⁺ EMP remained elevated after treatment with fluvastatin and Y-27632. CD62⁺ EMP was suppressed by 19% but this was not statistically significant. The total number of EMP specific for CD106 increased after treatment with fluvastatin and Y-27632, however this was not statistically significant.

Role of Rho/ROK

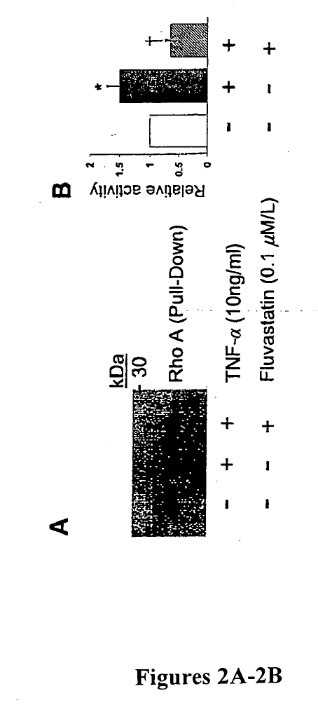

Inactivation of Rho GTPases by stains is known to regulate the expression of several endothelial genes whose products play key roles in thrombosis and vascular biology.[13] TNF-α (10 ng/mL) induced RhoA in activation in HCAEC at 24 hours. Fluvastatin (0.1 μM/L) significantly prevented TNF-α-induced RhoA activation (FIG. 2).

TABLE 1

| | | | TNF-α/ | TNF-α/ |
|---|---|---|---|---|
| | Control | TNF-α | Flu | Y-27632 |
| CD31 (PECAM-1) | 145 ± 36 | 233 ± 77* | 231 ± 80 | 255 ± 104 |
| CD105 (Endoglin) | 43 ± 5 | 124 ± 34* | 45 ± 8† | 56 ± 13‡ |
| CD51(Vitronectin | 29 ± 4 | 76 ± 46* | 43 ± 7† | 44 ± 10‡ |
| CD 54 (ICAM-1) | 82 ± 15 | 129 ± 44 | 125 ± 35 | 159 ± 24 |
| CD62E (ELAM) | 86 ± 2 | 99 ± 3 | 80 ± 7 | 65 ± 13 |
| CD106 (VCAM-1) | 128 ± 21 | 109 ± 41 | 167 ± 45 | 228 ± 80 |

Fluvastatin suppresses EMP release in activated HCAEC.
*p < 0.05 between TNF-α treatment and control.
†p < 0.05 between TNF-α and TNF-α plus Fluvastatin treatment.
‡p < 0.05 between TNF-α and TNF-α plus Y-27632 The data are of six individual experiments and are represented as the Mean ± SEM of absolute count of EMP (×10⁶/mL). Results are shown after 24 hours of no treatment or TNF-α plus Fluvastatin 0.1 μM/L or TNF-α plus Y-27632.

### REFERENCES

[1] Creager M A, Luscher T F, Cosentino F, Beckman J A. Diabetes and vascular disease: pathophysiology, clinical consequences, and medical therapy: Part I. Circulation. 108 (2003)1527-1532.
[2] Bernal-Mizrachi L, Jy W, Jimenez J J, Pastor J, Mauro L M, Horstman L L, de Marchena E, Ahn Y S. High levels of circulating endothelial microparticles in patients with acute coronary syndromes. Am Heart J. 145 (2003) 962-970.
[3] Mallat Z, Benamer H, Hugel B, Benessiano J, Steg P G, Freyssinet J M, Tedgui A. Elevated levels of shed membrane microparticles with procoagulant potential in the peripheral circulating blood of patients with acute coronary syndromes. Circulation. 101 (2000) 841-843.
[4] Jimenez J J, Jy W, Mauro L M, Soderland C, Horstman L L, Ahn Y S. Endothelial cells release phenotypically and quantitatively distinct microparticles in activation and apoptosis. Thromb Res. 109 (2003) 175-180.
[5] Downs J R, Clearfield M, Weis S, Whitney E, Shapiro D R, Beere P A, Langendorfer A, Stein E A, Kruyer W, Gotto A M Jr. Primary prevention of acute coronary events with lovastatin in men and women with average cholesterol levels: results of AFCAPS/TexCAPS. Air Force/Texas Coronary Atherosclerosis Prevention Study. JAMA. 279 (1998) 1615-1622.
[6] Marz W, Koenig W. HMG-CoA reductase inhibition: anti-inflammatory effects beyond lipid lowering? J Cardiovasc Risk. 10 (2003) 169-179.
[7] Ridley A J. Rho GTPases and cell migration. J Cell Sci. 114 (2001) 2713-2722.
[8] Takemoto M, Liao J K. Pleiotropic effects of 3-hydroxy-3-methylglutaryl coenzyme a reductase inhibitors. Arterioscler Thromb Vasc Biol. 21 (2001) 1712-1719.
[9] Omori H, Nagashima H, Tsurumi Y, Takagi A, Ishizuka N, Hagiwara N, Kawana M, Kasanuki H. Direct in vivo evidence of a vascular statin: a single dose of cerivastatin rapidly increases vascular endothelial responsiveness in healthy normocholesterolaemic subjects. Br J Clin Pharmacol. 54 (2002) 395-399.
[10] Horstman L L, Jy W, Jimenez J J, Ahn Y S. Endothelial micoparticles as markers of endothelial dysfunction. Front. Biosci. 9 (2004) 1118-1135.
[11] Musial J, Undas A, Gajewski P, Jankowski M, Sydor W, Szczeklik A. Anti-inflammatory effects of simvastatin in subjects with hypercholesterolemia. Int J Cardiol. 77 (2001) 247-253
[12] Teupser D, Bruegel M, Stein O, Stein Y, Thiery J. HMG-CoA reductase inhibitors reduce adhesion of human monocytes to endothelial cells. Biochem Biophys Res Commun. 289 (2001) 838-844.
[13] Laufs U, Liao J K. Post-transcriptional regulation of endothelial nitric oxide synthase mRNA stability by Rho GTPase. J Biol Chem. 273 (1998) 24266-24271.
[14] Topol E J. Intensive Statin Therapy—A Sea Change in Cardiovascular Prevention. N Engl J Med. 350 (2004) 1562-1564.
[15] Rasmussen L M, Hansen P R, Nabipour M T, Olesen P, Kristiansen M T, Ledet T. Diverse effects of inhibition of 3-hydroxy-3-methylglutaryl-CoA reductase on the expression of VCAM-1 and E-selectin in endothelial cells. Biochem J. 360 (2001) 363-370.
[16] Price D T, Loscalzo J. Cellular adhesion molecules and atherogenesis. Am J Med. 107 (1999) 85-97.
[17] Kataoka C, Egashira K, Inoue S, Takemoto M, Ni W, Koyanagi M, Kitamoto S, Usui M, Kaibuchi K, Shimokawa H, Takeshita A. Important role of Rho-kinase in the pathogenesis of cardiovascular inflammation and remodeling induced by long-term blockade of nitric oxide synthesis in rats. Hypertension. 39 (2002) 245-250.
[18] Mallat Z, Gojova A, Sauzeau V, Brun V, Silvestre J S, Esposito B, Merval R, Groux H, Loirand G, Tedgui A. Rho-associated protein kinase contributes to early atherosclerotic lesion formation in mice. Circ Res. 93 (2003) 884-888.
[100] Fischetti F, Carretta R, Borotto G, Durigutto P, Bulla R, Meroni P L, Tedesco F. Fluvastatin treatment inhibits leucocyte adhesion and extravasation in models of complement-mediated acute inflammation. Clin Exp Immunol. 135(2004) 186-193.
[101] Mitani H, Egashira K, Ohashi N, Yoshikawa M, Niwa S, Nonomura K, Nakashima A, Kimura M. Preservation of endothelial function by the HMG-CoA reductase inhibitor fluvastatin through its lipid-lowering independent antioxidant properties in atherosclerotic rabbits. Pharmacology. 68 (2003) 121-130.
[102] Omi H, Okayama N, Shimizu M, Fukutomi T, Imaeda K, Okouchi M, Itoh M. Statins inhibit high glucose-mediated neutrophil-endothelial cell adhesion through decreasing surface expression of endothelial adhesion molecules by stimulating production of endothelial nitric oxide. Microvasc Res. 65 (2003) 118-124.

1. A method for determining the presence of clinical atherosclerosis in a patient, said method comprising:
(a) obtaining a plasma sample from a patient;
(b) centrifuging the plasma sample of step (a) in order to collect a pellet of debris containing endothelial microparticles (EMPs);
(c) resuspending the pelleted debris of step (b) with an appropriate buffer;
(d) adding both a labeled antibody against cellular adhesion molecules (CAMs) that are specific to EMPs and the resuspended pellet of step (c) to a container having a known number of solid surfaces wherein the solid surfaces are labeled with a fluorescent dye;
(e) performing FACScan flow cytometry on the sample of step (d) in order to calculate the absolute number of EMPs therein; and
(f) correlating an increased level of EMPs in the sample derived from the patient, compared to a corresponding control sample, with the presence of clinical atherosclerosis.

2. A method for measuring atherosclerotic burden in a patient diagnosed with atherosclerosis, said method comprising:
(a) obtaining a plasma sample from a patient;
(b) centrifuging the plasma sample of step (a) in order to collect a pellet of debris containing endothelial microparticles (EMPs);
(c) resuspending the pelleted debris of step (b) with an appropriate buffer;
(d) adding both a labeled antibody against cellular adhesion molecules (CAMs) that are specific to EMPs and the resuspended pellet of step (c) to a container having a known number of solid surfaces wherein the solid surfaces are labeled with a fluorescent dye; and
(e) performing FACScan flow cytometry on the sample of step (d) in order to calculate the absolute number of EMPs therein.

3. The method of claim 1 or 2 wherein the clinical atherosclerosis is at least one of diabetes mellitus, coronary artery disease (CAD), or acute coronary syndrome.

4. The method of claim 1 or 2 wherein the plasma is ultra-centrifuged in a range of from about 15,000 to about 20,000×g.

5. The method of claims 1 or 2 wherein the buffer is PBS.

6. The method of claims 1 or 2 wherein the labeled antibodies are FITC-conjugated and/or PE-conjugated.

7. The method of claims 1 or 2 wherein the solid surfaces are beads.

8. The method of claim 7 wherein the size of the beads in the container are larger than 2 μM in diameter.

9. The method of claim 8 wherein the beads are between 3 and 5 μM in diameter.

10. The method of claim 9 wherein the beads are about 4 μM in diameter.

11. The method of claims 1 or 2 wherein the EMPs have at least one of CD62E, CD106, CD31, CD51, CD54, or CD105 antigens, and do not have the CD42a or CD42b antigen.

12. The method of claims 1 or 2 wherein the flow cytometry is two-color flow cytometry.

13. The method of claims 1 or 2 wherein the container having a known number of solid surfaces labeled with a binding partner to the labeled antibody is a TruCount™ tube.

14. The method of claims 1 or 2 wherein the fluorescent dye is PerCP.

15. The method of claim 7 wherein the beads are glass, plastic, acrylic, or polystyrene.

16. A kit for determining the presence of atherosclerosis in a patient or for measuring atherosclerotic burden in a patient, the kit comprising: a labeled antibody against one or more cellular adhesion molecules (CAMs) specific to EMPs, and a container such as a tube containing a known number of sold surfaces wherein the sold surfaces are labeled with a fluorescent dye.

17. The kit of claim 16 further comprising at least one of: a container for collecting a blood from a patient, a container for preparing a plasma sample, or an appropriate buffer for resuspending debris pelleted from the plasma sample.

18. The kit of claim 16 further comprising instructions for its use.

19. The kit of claim 16 or 17 wherein the labeled antibody is at least one of monoclonal anti-human CD62E-FITC, CDC105-FITC, CD51-FITC, CD106-PE, CD31-PE, or CD54-PE.

20. The method of claim 1 or 2 wherein the labeled antibody is at least one of monoclonal anti-human CD62E-FITC, CDC105-FITC, CD51-FITC, CD106-PE, CD31-PE, or CD54-PE.
